(19) United States
(12) Patent Application Publication
(10) Pub. No.: US 2007/0292477 A1
(43) Pub. Date: Dec. 20, 2007

(54) Association of Antimicrobial Compounds with Surfaces and Polymers

(76) Inventor: Naresh Kumar, Maroubra (AU)

Correspondence Address: NIXON & VANDERHYE, PC, 901 NORTH GLEBE ROAD, 11TH FLOOR, ARLINGTON, VA, 22203, US

(21) Appl. No.: 10/581,710

(22) PCT Filed: Dec. 6, 2004
(86) PCT No.: PCT/AU04/01703

§ 371 Date: Mar. 28, 2007

(30) Foreign Application Priority Data
Dec. 5, 2003 (AU) 2003906771

(51) Int. Cl. A61K 31/765; A61K 31/787; A61K 9/00; A61L 15/20; A61M 25/00; A61P 31/00; C07D 207/24; C07D 307/26

(52) U.S. Cl. 424/429; 424/447; 424/78.3; 424/78.36; 424/78.38; 548/543; 549/479; 604/265

(57) ABSTRACT

The invention describes antimicrobial silicone oligomers or polymers comprising a silicone oligomer or polymer associated with at least one compound of formula (I). Also described are methods of associating compounds of formula (I) to surfaces and products thereof.

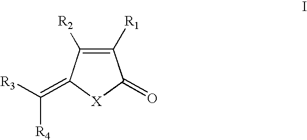

### FIELD OF THE INVENTION

This invention relates to antimicrobial polymers. In particular, this invention relates to silicone polymers (e.g. silanes, siloxanes, silicone rubbers etc) associated with antimicrobial compounds. The invention further relates to the attachment of antimicrobial compounds to surfaces.

### BACKGROUND OF THE INVENTION

Fimbrolides (halogenated 5-methylene-2(5H)-furanones) possess a wide range of important biological properties including antifungal and antimicrobial properties. These metabolites can be isolated from red marine algae Delisea fimbriata, Delisea elegans and Delisea pulchra, and a variety of structurally related furanones (e.g. degree and position of substitution of halogen on the furanone ring system, type of heteroatoms present in the furanone ring, and the length and position of the sidechain with respect to the furanone carbonyl group) can be derived through synthesis. Halogenated furanones regulate the phenotypes of Gram positive and Gram negative bacteria, and interfere with their settlement and motility on treated surfaces (see for example PCT/AU95/00407, PCT/AU96/00167, PCT/AU99/00285, PCT/AU99/00284, PCT/AU00/01553, PCT/AU01/00296, PCT/AU01/00295, PCT/AU01/00407, PCT/AU01/00781 and PCT/AU01/01621, the disclosures of which are incorporated herein in their entirety by cross-reference)

Lactam analogues of fimbrolides have also been shown to possess antimicrobial properties (see for example, PCT/AU03/01053, the disclosure of which is incorporated herein in its entirety by cross reference).

Bacterial biofilms are undesirable on many types of surfaces. Such surfaces include, for example, cooling water towers, household and industrial surfaces, pipes, membranes, hospital surfaces, food preparation surfaces, packaging, biomedical devices and electronic devices. Currently chemical biocides such as bleach, ammonia, quaternary ammonium salts and strong alkaline solutions are used to remove such biofilms. These chemical biocides may have a harmful effect on the environment. Therefore, the use of naturally derived antimicrobial compounds or derivatives thereof are becoming increasingly desirable and necessary.

The present inventors have found that furanone compounds and the like can be incorporated into silicone polymers and these polymers can be used in protecting a wide range of devices and equipment from biological damage due to Gram-negative and Gram-positive bacteria.

### SUMMARY OF THE INVENTION

In a first aspect the present invention provides an antimicrobial silicone oligomer or polymer associated with at least one compound of formula I 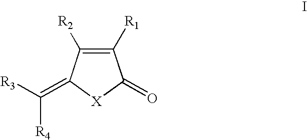
wherein, R₁ and R₂ are independently selected from the group consisting of H, halogen, alkyl, alkoxy, oxoalkyl, alkenyl, aryl or arylalkyl whether unsubstituted or substituted, straight chain or branched chain, hydrophobic, hydrophilic or fluorophilic;
R₃ and R₄ are independently H, halogen, alkyl, aryl, or arylalkyl; and
X is O or NR₂.

Preferably, at least one of R₁, R₂, R₃ and R₄ is a halogen. More preferably, at least one of R₁, R₂, R₃ and R₄ is bromine. The compound of formula I may be a furanone or a lactam.

The antimicrobial oligomer or polymer may be formed by addition or condensation oligomerisation or polymerisation. The silicone oligomer or polymer may be a linear, cross-linked, or a cyclic polymer.

The antimicrobial oligomer or polymer may be in the form of a fluid, for example, an oil which may have a wide range of chain lengths and molecular masses, depending on the particular application. Examples of such applications are cooling and dialectric fluids, in polishes and waxes, as release and antifoam agents, and for paper and textile treatment.

It may be, or form part of, a resin forming composition, for example, for use in or as a hard coating, film or paint. It may be suitable for use in an adhesive.

The antimicrobial oligomer or polymer of the invention may be in the form of a gel having lightly cross-linked polysiloxane networks. The degree of cross-linking may be selected to achieve the desired physical properties of the gel.

The antimicrobial oligomer or polymer may be an elastomer or rubber. The elastomer or rubber may be extensively cross-linked. The antibacterial oligomer or polymer of the present invention may be, or form part of, a curable or vulcanisable composition. Silicone elastomers may be high molecular weight linear polymers. These can be cured by a number of ways, for example, by free radical cross linking (eg using benzoyl peroxide) to form bridges between the chains; by crosslinking vinyl groups attached to silicon through reaction with silylhydride groups; by crosslinking linear or branched siloxane chains having reactive end groups such as silanols to yield Si—O—Si crosslinks. These materials have outstanding low temperature flexibility, are stable at high temperatures and are resistant to weathering and lubricating oils. They may be used in gaskets and seals, wire and cable insulation, and hot gas and liquid conduits. They also have application in surgical and prosthetic devices. Curable room temperature vulcanising silicone elastomers have application in caulking, sealing and encapsulating.

The polymer or oligomer may form the whole, or part, of a shaped article. For example, the oligomer or polymer may be cured, cast or extruded to form a desired shape or a device. The antibacterial oligomer or polymer of the present invention may be, or comprise at least part of, a film or sheet.

In a further aspect, the present invention provides a compound of formula III: 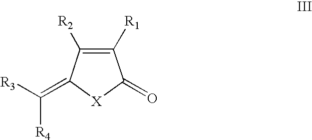
wherein, R₁ and R₂ are independently selected from the group consisting of H, halogen, alkyl, alkoxy, oxoalkyl, alkenyl, aryl or arylalkyl whether unsubstituted or substituted, straight chain or branched chain, hydrophobic, hydrophilic, or fluorophilic,
R₃ and R₄ are independently H, halogen, alkyl, aryl, or arylalkyl; and
X is O or NR₂,
wherein the compound of formula III has at least one —YC(O)NR₇R₅Si(OR₆)₃ group, where Y is selected from the group O, S, N, P, C(O); R₅ is a linker and preferably is substituted or unsubstituted alkyl, alkylaryl, arylalkyl, aryl, alkenyl, or a linker comprising these groups, optionally interrupted by one of more heteroatoms (eg oxygen), or a linking group comprising these groups and each R₆ is independently selected from substituted or unsubstituted alkyl, cycloalkyl, alkenyl or the like and R₇ is H or alkyl.

Methods of preparing compounds of formula III and attaching compounds of formula III to surfaces such as glass, silicone rubber or polymeric surfaces are also contemplated.

### BRIEF DESCRIPTION OF THE FIGURES

FIG. 1 shows compounds and corresponding hydroxylated derivatives for use in one embodiment of the present invention.

FIG. 2 shows biofilm formation by P. aeruginosa on furanone treated surfaces according to the present invention under conditions of flow.

FIG. 3 shows biofilm formation by S. aureus on furanone treated catheters according to the present invention.

### DETAILED DESCRIPTION OF THE INVENTION

In one embodiment of the present invention, the antimicrobial silicone oligomer or polymer comprises a compound of formula I blended or mixed therewith, 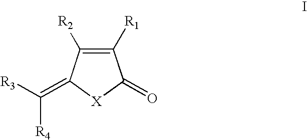
wherein, R₁ and R₂ are independently H, halogen, alkyl, alkoxy, oxoalkyl, alkenyl, aryl or arylalkyl whether unsubstituted or substituted, straight chain or branched chain, hydrophilic or fluorophilic;
R₃ and R₄ are independently H, halogen, alkyl, aryl or arylalkyl; and
X is O or NR₂.

Preferably at least one of R₁, R₂, R₃ and R₄ is halogen, most preferably bromine.

The silicone oligomer or polymer can be a linear or cross-linked polymer, or a cyclic polymer. Non-limiting examples of silicone oligomer or polymer include hexamethyldisiloxane, octamethyltrisiloxane, decamethyltetrasiloxane, dodecamethylpentasiloxane, tetradecamethylhexasiloxane, hexamethyltricyclosiloxane, decamethylpentacyclosiloxane, dodecamethylhexacyclosiloxane, dimethylpolysiloxane.

It is known in the art that the silicone rubber can be fabricated by a molding process (including liquid injection molding, transfer and compression molding) or an extrusion process by mixing and curing silicone with a catalyst and filler. In this invention the furanone can be optionally mixed with silicone or a cross linker or catalyst during the production of silicone oligomer or polymer. The polymer may be a homopolymer or copolymer.

In a further embodiment, the present invention provides an oligomer or polymer according to the first aspect of invention, wherein the compound of formula I is adsorbed to the polymer or oligomer, 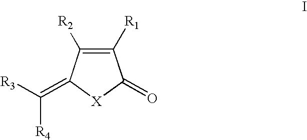
wherein X, R₁, R₂, R₃ and R₄ are as described above.

The compound of formula I may be adsorbed to the silicone polymer by direct application to the compound of formula I to the polymer. For example a material or device having at part of its surfaced formed from the polymer may be treated by either dip coating or painting the surface of the device with a solution of compound. The device or material may be a molded, extruded or assembled device. Examples of devices or materials include catheters, drain and fluid tubes, sheathing, shunts, pulmonary devices, laparoscopic devices, occluders, ear plugs, hearing aids, seals/stoppers/valves, septums, valves, contact lenses, orthopaedic implants, membranes, pipes, tubing, tanks, etc.

In a further aspect the present invention provides an antimicrobial silicone oligomer or polymer formed by copolymerising a compound of formula I 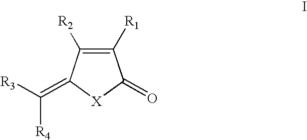
with at least one silicone comonomer or oligomer and optionally at least one other monomer,
wherein R₁ and R₂ are independently H, halogen, alkyl, alkoxy, oxoalkyl, alkenyl, aryl or arylalkyl whether unsubstituted or substituted, straight chain or branched chain, hydrophilic or fluorophilic;
R₃ and R₄ are independently H, halogen, alkyl, aryl or arylalkyl; and
X is O or NR₂.

Preferably, at least one of R₁, R₂, R₃ and R₄ is halogen.

Non-limiting examples of silicone oligomer or polymer include hexamethyldisiloxane, octamethyltrisiloxane, decamethyltetrasiloxane, dodecamethylpentasiloxane, tetradecamethylhexasiloxane, hexamethyltricyclosiloxane, decamethylpentacyclosiloxane, dodecamethylhexacyclosiloxane, dimethylpolysiloxane.

Preferably the compound of the formula I is a compound of formula II 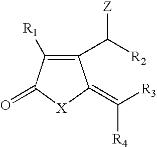
wherein R₁, R₂ are independently selected from H, alkyl, alkoxy, polyethyleneglycol, oxoalkyl, alkenyl, aryl or arylalkyl whether unsubstituted or substituted, straight chain or branched chain, hydrophobic, hydrophilic or fluorophilic;
R₄ is a hydrogen, halogen (X═F, Cl, Br or I);
R₃ is hydrogen or halogen; and
X is O or NR₂ and
Z is independently selected from the group R₂, halogen, OH, OOH, OC(O)R₂, ═O, amine, azide, thiol, mercaptoalkyl, mercaptoalkenyl, alkenyloxy, aryloxy, mercaptoaryl, arylalkoxy, mercaptoarylalkyl, SC(O)R₂, OS(O)₂R₂, NHC(O)R₂, ═NR₂, NHR₂ or silyloxy.

In a yet a further embodiment, the present invention provides an antimicrobial silicone oligomer or polymer formed by condensation polymerisation of a silicone monomer or oligomer or polymer with a compound of formula I 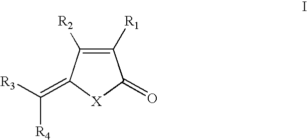
wherein R₁ and R₂ are independently H, halogen, alkyl, alkoxy, oxoalkyl, alkenyl, aryl or arylalkyl whether unsubstituted or substituted, straight chain or branched chain, hydrophilic or fluorophilic;
R₃ and R₄ are independently H, halogen, alkyl, aryl or arylalkyl;
X is O or NR₂.

Preferably, at least one of R₁, R₂, R₃ and R₄ is halogen.

The silicone oligomer or polymer can be a linear or cross-linked polymer, or a cyclic polymer. Examples of silicone oligomer or polymer include hexamethyldisiloxane, octamethyltrisiloxane, decamethyltetrasiloxane, dodecamethylpentasiloxane, tetradecamethylhexasiloxane, hexamethyltricyclosiloxane, decamethylpentacyclosiloxane, dodecamethylhexacyclosiloxane, dimethylpolysiloxane.

Preferably the compound of the formula I is a compound of formula II; 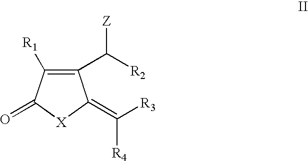
wherein R₁, R₂ is a H, alkyl, alkoxy, polyethyleneglycol, oxoalkyl, alkenyl, aryl or arylalkyl whether unsubstituted or substituted, straight chain or branched chain, hydrophobic, hydrophilic or fluorophilic;
R₄ is a hydrogen, halogen (X═F, Cl, Br or I);
R₃ are independently or both hydrogen or halogen;
X is O or NR₂ and
Z is independently selected from the group R₂, halogen, OH, OOH, OC(O)R₂, ═O, amine, azide, thiol, mercaptoalkyl, mercaptoalkenyl, alkenyloxy, aryloxy, mercaptoaryl, arylalkoxy, mercaptoarylalkyl, SC(O)R₂, OS(O)₂R₂, NHC(O)R₂, ═NR₂, NHR₂ or silyloxy.

In yet a further embodiment, the present invention provides an antimicrobial silicone polymer formed by surface attachment of a compound of formula I on to a silicone polymer or a device formed at least in part therefrom 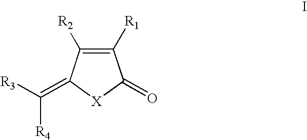
wherein R₁ and R₂ are independently H, halogen, alkyl, alkoxy, oxoalkyl, alkenyl, aryl or arylalkyl whether unsubstituted or substituted, straight chain or branched chain, hydrophilic or fluorophilic;
R₃ and R₄ are independently H, halogen, alkyl, aryl or arylalkyl; and
X is O or NR₂.

Preferably at least one of R₁, R₂, R₃ and R₄ is halogen.

It is known in the art that the surface attachment of a compound to a polymer surface may require the initial activation of a surface by chemical means or plasma activation. The silicone polymers or devices used in this invention may be optionally chemically or plasma treated.

Preferably the compound of the formula I is of formula II, 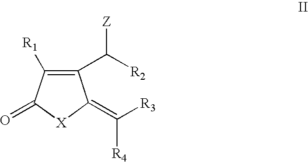
wherein R₁, R₂ is a H, alkyl, alkoxy, polyethyleneglycol, oxoalkyl, alkenyl, aryl or arylalkyl whether unsubstituted or substituted, straight chain or branched chain, hydrophobic, hydrophilic or fluorophilic;
R₄ is a hydrogen, halogen (X═F, Cl, Br or I);
R₃ are independently or both hydrogen or halogen;
X is O and NR₂ and
Z is independently selected from the group R₂, halogen, OH, OOH, OC(O)R₂, ═O, amine, azide, thiol, mercaptoalkyl, mercaptoalkenyl, alkenyloxy, aryloxy, mercaptoaryl, arylalkoxy, mercaptoarylalkyl, SC(O)R₂, OS(O)₂R₂, NHC(O)R₂, ═NR₂, NHR₂ or silyloxy.

In yet a further aspect, the present invention includes a shaped article or a device formed at least in part from an antimicrobial polymer or oligomer of the present invention. The shaped article or device may be formed by curing, casting or extruding the polymer to a desired shape or a device.

As will be recognised by those skilled in the art, compounds of formulae I and II can exist in two isomer forms. It is not intended that the compounds of formulae I and II be limited to any particular isomer and so the present invention extends to all isomers of the compounds defined by the formulae.

The present invention also extends to methods of making the antimicrobial polymers and oligomers of the invention.

In a further aspect, the present invention provides a compound of formula III that is of formula 1 and having at least one —YC(O)NR₇R₅Si(OR₆)₃ group, where Y is selected from the group O, S, N, P, C(O); R₅ is a linker and preferably is substituted or unsubstituted alkyl, alkylaryl, arylalkyl, aryl, alkenyl, or a linker comprising these groups, optionally interrupted by one of more heteroatoms (eg oxygen), or a liking group comprising these groups and each R₆ is independently selected from substituted or unsubstituted alkyl, cycloalkyl, alkenyl or the like and R₇ is H or alkyl.

Accordingly, the present invention provides a compound of formula III: 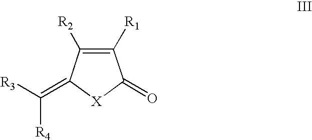
wherein, R₁ and R₂ are independently selected from the group consisting of H, halogen, alkyl, alkoxy, oxoalkyl, alkenyl, aryl or arylalkyl whether unsubstituted or substituted, straight chain or branched chain, hydrophobic, hydrophilic, or fluorophilic,
R₃ and R₄ are independently H, halogen, alkyl, aryl, or arylalkyl; and
X is O or NR₂,
wherein the compound of formula III has at least one —YC(O)NR₇R₅Si(OR₆)₃ group, where Y is selected from the group O, S, N, P, C(O); R₅ is a linker and preferably is substituted or unsubstituted alkyl, alkylaryl, arylalkyl, aryl, alkenyl, or a linker comprising these groups, optionally interrupted by one of more heteroatoms (eg oxygen), or a linking group comprising these groups and each R₆ is independently selected from substituted or unsubstituted alkyl, cycloalkyl, alkenyl or the like and R₇ is H or alkyl.

Preferably, R₅ is a polyoxoalkylene. More preferably, R₅ is a polyethylene glycol of molecular weight from 400 to 4000.

In a preferred embodiment, R₁ or R₂ is hydrophilic. Hydrophilic substituents provide advantages when devices coated with a compound of formula III are inserted into physiological environments.

In another preferred embodiment, R₁ or R₂ is hydrophobic. Hydrophobic substituents provide advantages when devices, such as bandages, coated with a compound of formula III are used eg in wound care applications.

In a yet further preferred embodiment, R₁ or R₂ is fluorophilic. Compounds comprising fluorophilic side-chains can provide air permeability to surfaces coated therewith which provides advantages when devices coated with a compound of formula III are used in certain wound care applications.

In a further aspect, the present invention provides a method of producing a compound according to formula III, comprising reacting a compound of formula I having at least one group selected from —Y′—H, wherein —Y′ is selected from the group O, S, NH, COO with a compound of formula OCNR₇R₅Si(OR₆)₃, wherein R5 and R6 are as defined above.

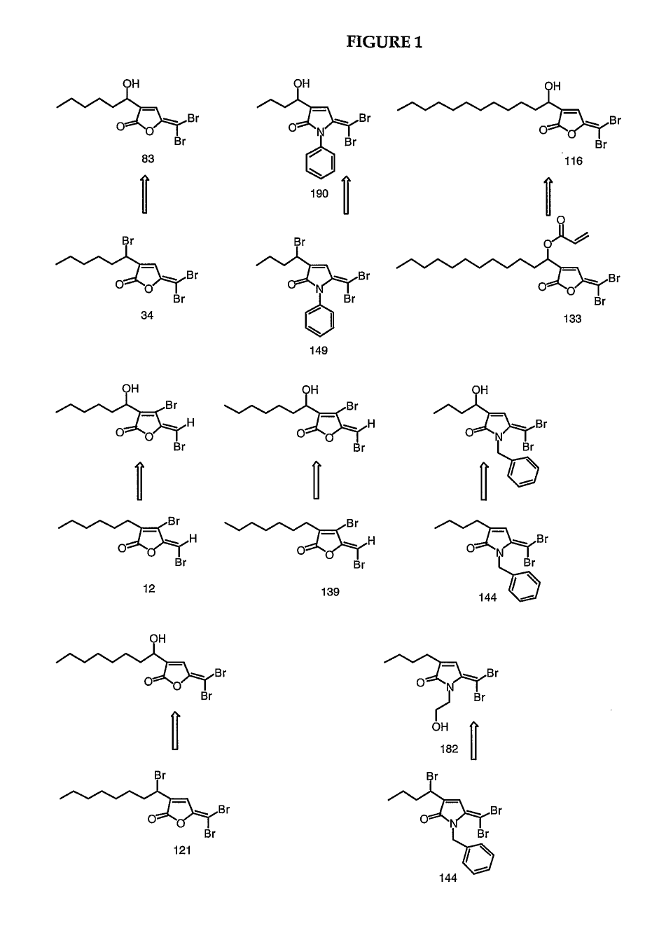

Preferably, the group —Y′H is a hydroxyl group. Compounds of formula I which comprise hydroxyl groups can be synthesised by techniques known in the art such as those disclosed in PCT/AU99/00285. FIG. 1 illustrates a number of furanones and lactams of formula I and their hydroxylated derivatives. Further compounds suitable for use in the present invention include:

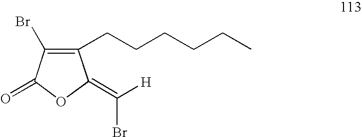

In yet a further aspect, the present invention provides a method for associating a compound of formula III with a surface, the method comprising contacting the compound of formula III with the surface and optionally curing the compound.

The surface may be treated to produce groups that are reactive with the silyloxy group of the compound of formula III.

The compound of formula III may also be associated with an oligomer or polymer as described. The oligomer or polymer may be an antimicrobial silicone oligomer or polymer as described above. Alternatively the compound of formula III, may be associated with a non-silicone oligomer or polymer, surface or device. The present invention also extends to an oligomer or polymer associated with a compound of formula III.

The antimicrobial polymers of the present invention have application in those applications in which silicone polymers or oligomers are used. Silicone polymers are incorporated into medicines; used in food processing (for example, canning and ready meals); used in a wide range of medical devices; used as putty and sealants; used as membrane pipes and tubing.

Silicone is also used in domestic and personal products such as cleaning solvents, plumbing, handcream, hair and skin products, and antiperspirants.

The antimicrobial silicone polymers and oligomers of the present invention may be suitable for the following non-limiting applications—silicone impregnated electrical insulating tapes, silicone rubber, adhesives, sealants, elastoplastic resins for coatings of circuit boards, compounds for potting and protecting semiconductor devices, dielectric compounds, high-purity coatings, varnishes, resins, specialty lubricants, optical fibre coatings and fibre optic cable filler, and semiconductor-grade silicon and silicon-source chemicals, windshield and canopy gasket sealants, rubber tooling for radome fabrication, optical interlayer laminates, abrasion-resistant coatings, adhesives, seals and gaskets, and tooling materials, construction adhesive/sealants, glazing adhesive/sealants and elastomers, silicone/polyurethane foam roof coatings, fire retardant foams and sealants, architectural coatings and water repellents, concrete pavement joint sealants, antifoams, bakeware coatings; processing aids for food processing applications; automotive applications including heat, oil and fuel-resistant silicone rubbers; one or two part sealants and adhesives, specialty lubricants and materials for noise, vibration, harshness and thermal management, automotive polishes; adhesives including silicone elastomers, adhesives, sealants, dielectric compounds, varnishes, multi-purpose silicone fluids, antifoams, release agents, surfactants, maintenance lubricants, elastomers and greases; medical applications and medical products including medical-grade tubing, adhesives, defoamers and fluids; textiles and leather applications, for example, waterproofing treatments and fibre chemicals; silicone adhesives, sealants and caulks for home improvement and renovation by do-it-yourselves; paints and coatings applications, for example silicone additives for high-performance paints, enamels, finishes and abrasion resistant coatings for plastics; silicone rubber compounds for temperature and chemical resistant printing equipment component; applications in plastics, for example, mold release additives, catalyst modifiers, and chemicals for high-performance plastics applications; pressure sensitive adhesives, for example, release coatings for backings on tapes, labels, stamps, stickers, decals and food packaging; pressure-sensitive adhesives; personal and household care, for example, surfactants, emulsions, fluids and powder treatments are important ingredients in skin and suntan lotions, antiperspirants and deodorants, hair care products, shaving creams, cosmetics, starches, fabric treatments, laundry products hair care products; pharmaceutical applications, for example, medical-grade fluids, emulsions, antifoams, adhesives and silicone rubber tubing; medical devices, for example, heart valves, contact lenses, surgical equipment, catheters, drain and fluid tubes, sheathing, shunts, pulmonary devices, laparoscopic devices, occluders, ear plugs, hearing aids, seals/stoppers/valves, septums etc temporomandibular joint (jaw) implants; small joint orthopaedic (finger) implants; large joint products (hip, knee, elbow) implants; long term implantable contraceptives; silicone fluids for injection and certain custom silicone implant products; cleaning applications including toilet cleaners and industrial cleaning agents, membranes, water filters, air conditioning and cooling towers; material used in dentistry, such as dentures and false teeth.

Compounds of formula III may be attached to any surface regardless of whether that surface comprises silicone polymers. In circumstances where the surface does not comprise groups reactive with a sillyloxy group then these groups can be generated by techniques known to those skilled in the art eg plasma activation techniques. Surfaces to which compounds of formula III are attached may be suitable for the following non-limiting applications: medical devices, for example, heart valves, contact lenses, surgical equipment, catheters, drain and fluid tubes, sheathing, shunts, pulmonary devices, laparoscopic devices, occluders, ear plugs, hearing aids, seals/stoppers/valves, septums etc temporomandibular joint (jaw) implants; small joint orthopaedic (finger) implants; large joint products (hip, knee, elbow) implants; long term implantable contraceptives; alginate beads.

Definitions

The term “alkyl” is taken to include both straight chain alkyl groups such as methyl, ethyl, propyl, isopropyl, butyl, isobutyl, sec-butyl, tertiary butyl, and the like. Preferably the alkyl group is a lower alkyl of 1 to 6 carbon atoms. The alkyl group may optionally be substituted by one or more groups selected from alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocyclyl, heterocycloxy, heterocyclamino, haloheterocyclyl, alkylsulfenyl, alkylcarbonyloxy, alkylthio, acylthio, silyloxyalkyl, phosphorus-containing groups such as phosphono and phosphinyl.

The term “alkoxy” includes straight chain or branched alkyloxy, preferably C1-10 alkoxy. Examples include methoxy, ethoxy, n-propoxy, isopropoxy and the different butoxy isomers.

The term “alkenyl” includes groups formed from straight chain, branched or mono- or polycyclic alkenes and polyene. Substituents include mono- or poly-unsaturated alkyl or cycloalkyl groups as previously defined, preferably C2-10 alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, or 1,3,5,7-cyclooctatetraenyl.

The term “halogen” denotes fluorine, chlorine, bromine or iodine, preferably bromine or fluorine.

The term “heteroatoms” denotes O, N or S.

The term “acyl” used either alone or in compound words such as “acyloxy”, “acylthio”, “acylamino” or diacylamino” includes an aliphatic acyl group and an acyl group containing a heterocyclic ring which is referred to as heterocyclic acyl, preferably a C1-10 alkanoyl. Examples of acyl include carbamoyl; straight chain or branched alkanoyl, such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heptyloxycarbonyl; cycloalkanecarbonyl such as cyclopropanecarbonyl cyclobutanecarbonyl, cyclopentanecarbonyl or cyclohexanecarbonyl; alkanesulfonyl, such as methanesulfonyl or ethanesulfonyl; alkoxysulfonyl, such as methoxysulfonyl or ethoxysulfonyl; heterocycloalkanecarbonyl; heterocyclyoalkanoyl, such as pyrrolidinylacetyl, pyrrolidinylpropanoyl, pyrrolidinylbutanoyl, pyrrolidinylpentanoyl, pyrrolidinylhexanoyl or thiazolidinylacetyl; heterocyclylalkenoyl, such as heterocyclylpropenoyl, heterocyclylbutenoyl, heterocyclylpentenoyl or heterocyclylhexenoyl; or heterocyclylglyoxyloyl, such as, thiazolidinylglyoxyloyl or pyrrolidinylglyoxyloyl.

The term “substituted” includes substitution by one or more groups selected from alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocyclyl, heterocycloxy, heterocyclamino, haloheterocyclyl, alkylsulfenyl, alkylcarbonyloxy, alkylthio, acylthio, phosphorus-containing groups such as phosphono and phosphinyl; —YC(O)NR₅Si(OR₆)₃, where Y is selected from the group O, S, N, P, C(O), R₅ is a linker group which may be, for example, substituted or unsubstituted alkyl, alkylaryl, arylalkyl, aryl, alkenyl, optionally interrupted by one of more heteroatoms and each R₆ is independently selected from substituted or unsubstituted alkyl, cycloalkyl, alkenyl or the like.

The term “fluorophilic” is used to indicate the highly attractive interactions that certain groups, such as highly fluorinated alkyl groups of C4-C10 chain length, have for perfluoroalkanes and perfluoroalkane polymers.

The one or more other monomer may be any suitable polymerisable copolymer e.g. acrylate ester such as alkyl, hydroxyalkyl, aminoalkyl, or substituted aryl acrylates or methacrylates, crotonates, substituted or unsubstituted acrylonitriles, vinyl alcohols or acetates, styrene and siloxanes.

The present invention will now be described with reference to the following non-limiting examples of the invention.

### EXAMPLE 1

A siloxane polymer with adsorbed furanone is prepared by soaking a silicone polymer or a device in a solution of 3-(1′-bromooctoyl)-4-bromo-5-bromomethylene-2(5H)-furanone, in an organic solvent.

### EXAMPLE 2

A condensation siloxane polymer is synthesised by heating a mixture of poly-dimethylsiloxane (PDMS) and 3-(1′-hydroxydodecyl)-5-dibromomethylene-2(5H)-furanone.

### EXAMPLE 3

A copolymerised siloxane polymer is synthesised by heating a mixture of methyl methacrylate (MMA), 3-(1′-acryloyloxydodecyl)-5-dibromomethylene-2(5H)-furanone, poly-dimethylsiloxane (PDMS) and (AIBN) in toluene at 70*C.

### EXAMPLE 4

A crosslinked siloxane polymer is synthesised by heating a mixture of 3-(1′-acryloyloxybutyl)-4-bromo-5-bromomethylene-2(5H)-furanone and poly-dimethylsiloxane (PDMS) in the presence of a platinum catalyst.

### EXAMPLE 5

A copolymerised siloxane polymer is synthesised by heating a mixture of styrene, 3-(1′-bromohexyl)-4-bromo-5-bromomethylene-2(5H)-furanone, poly-dimethylsiloxane (PDMS) and (AIBN) in toluene at 70° C.

### EXAMPLE 6

### General Procedure for Production of Compound III

A mixture of equivalent amount of furanone alcohol and silyloxypropyl isocyanate in dry dichloromethane was stirred at room temperature for 3 h. The resulting solution was concentrated and spin-coated on a glass or metal surface. The resulting film was cured either by heating at 120 degrees celcius or at room temperature for 24 h. The presence of furanone on the surface of the film was established by XPS analysis.

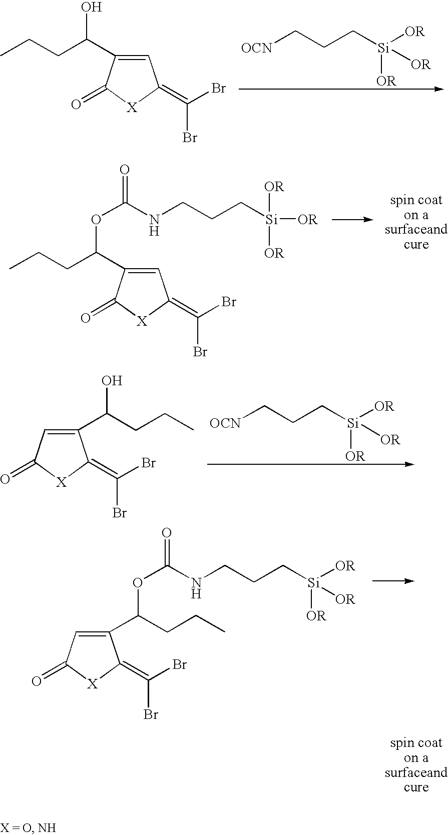

The alcohol group could be present any where in the molecule e.g.

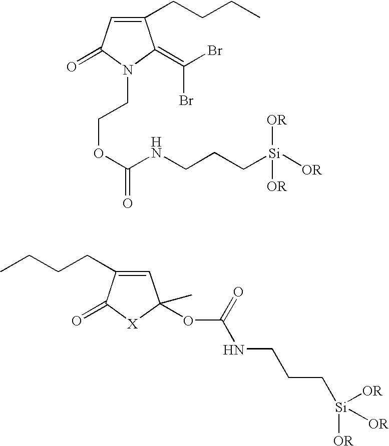

### EXAMPLE 7

Glass surfaces, catheters and contact lenses were surface modified by the covalent attachment of silyloxy derivatives of compounds 83, 116, 190 and 144 of FIG. 1 and compounds 135 and C6, depicted below, in accordance with the following general procedure. 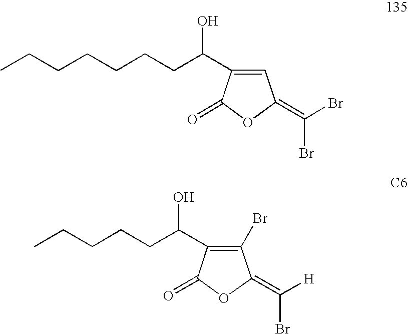
Attachment Using a Short Linker

Furanones containing a hydroxyl group e.g. 83, 116, 135, 190 and C6 were reacted with 3-isocyanatopropyltriethoxysilane to yield a carbamate linked furanone/lactam intermediate with a terminal triethoxysilane group. This intermediate was then coated onto the surface via either spin coating or spreading, and the resulting furanone/lactam layer was silanized by curing the glass at 90° C., a process that resulted in the reaction of the terminal triethoxysilane group with the surface hydroxyl groups (Scheme 1) thus leading to covalent attachment of the furanones/lactams. This technique is applicable to any surface that possesses surface hydroxyl groups.

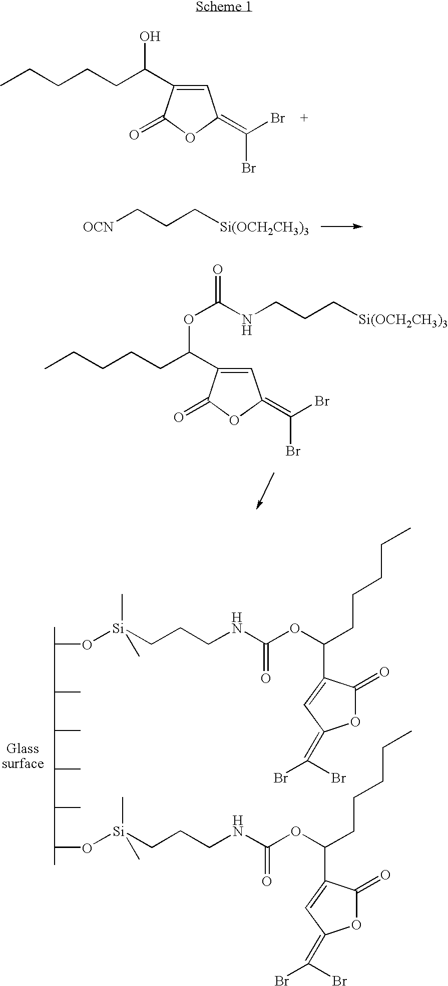

Lactam 144 was functionalised to the corresponding hydroxyl analogues prior to being attached to the surface as outlined above.

Attachment of via a PEG Linker

The presence of a linker group would raise the furanone/lactam higher off the surface of the biomaterial, which has the potential to make it more biologically available. Furthermore the presence of a suitable PEG linker would permit the furanone/lactam molecule to permeate the cell membrane. Therefore the furanones/lactams were also attached to the surfaces via a polyethylene glycol linker.

Initially one of the terminal hydroxyl groups of polyethylene glycol (MW 400 and MW 4000) was reacted with 3-isocyanatopropyltriethoxysilane to yield a PEG derivative with a terminal silane group on one end and a hydroxyl group on the other. The second hydroxyl group was then be reacted with bis-isocyanate to yield a PEG with isocyanate and silane terminal groups. The isocyanate end group was then be reacted with the furanone/lactam alcohol to form a carbamate link and the terminal silane group was used to covalently attach the furanone/lactam to the surface (Scheme 2). 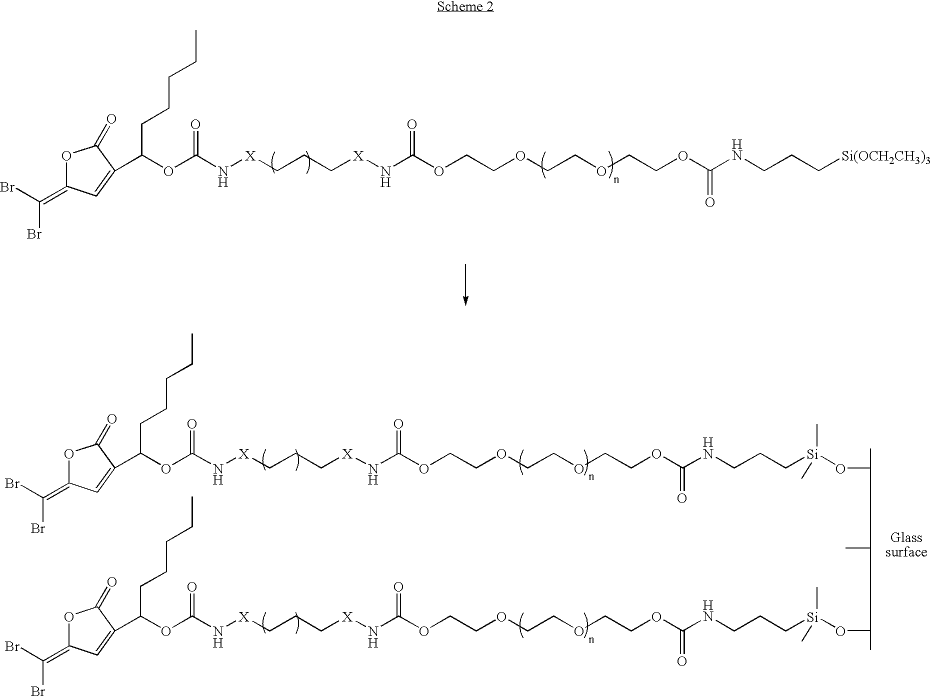
Attachment of Compounds onto Contact Lens Surfaces

The abovementioned methodologies were also investigated with contact lenses which already have hydroxyl group on their surfaces. The resulting lenses were then analysed by XPS to assess the efficiency of the attachment process.

Synthesis of Furanone/Lactam Analogues for Surface Attachment

### Reaction of Furanone 116 with (3-isocyanatopropyl)-triethoxysilane

Furanone 116 (1.04 g, 2.37 mmol) and (3-isocyanatopropyl)-triethoxysilane (1.5 mL, 6 mmol) were stirred together at 85° C. for 24 h. The excess isocyanate reactant was removed in vacuo at 50° C./0.2 mmHg. The residue was flash chromatographed through a short plug of silica gel with CH₂Cl₂/ethylacetate (29:1) as the eluent to give 1-(5,5-dibromomethylene-3-dodecyl-2(5H)furanone)triethoxysilylpropylcarbamate as a colourless oil (0.50 g, 100%). ¹H n.m.r. (300 MHz, CDCl₃) δ: 0.63; 0.86; 1.22; 1.60; 1.66; 3.17; 3.81; 4.10; 4.79; 5.48; 7.39; 7.49. ¹³C n.m.r. (75.5 MHz, CDCl₃) δ: 7.6; 14.0; 18.2; 22.6; 23.0; 24.5; 24.9; 25.0; 29.2; 29.5; 31.8; 35.5; 36.0; 43.4; 53.3; 58.4; 67.1; 67.6; 69.0; 80.3; 80.6; 81.0; 134.0; 134.5; 134.8; 136.7; 140.2; 149.3; 149.5; 155.2; 166.0; 166.6.

### Reaction of 1-hexanol with (3-isocyanatopropyl)-triethoxysilane

1-Hexanol (2 mL, 0.01 mol) and (3-isocyanatopropyl)-triethoxysilane (3.2 g, 0.13 mol) were stirred together at 85° C. for 24 h. The excess isocyanate was removed under high vacuum (50° C./0.2 mmHg) and the colourless carbamate derivative (3.6 g, 100%) was used without further purification. ¹H n.m.r. (300 MHz, CDCl₃) δ: 0.62; 0.88; 1.22; 1.30; 3.16; 3.81; 4.03; 4.85. ¹³C n.m.r. (75.5 MHz, CDCl₃) δ: 7.5; 13.9; 14.5; 18.1; 18.3; 22.4; 23.2; 25.4; 28.9; 31.4; 38.4; 43.3; 44.2; 47.2; 56.0; 58.3; 64.9; 68.3; 104.5; 156.8; 159.6.

The following silyl analogues were similarly prepared.

Furanone C6Silyl Compound

¹H n.m.r. (300 MHz, CDCl₃) δ: 0.60; 1.23; 1.62; 3.14; 3.73; 4.10; 5.47; 6.36. ¹³C n.m.r. (75.5 MHz, CDCl₃) δ: 7.5; 13.8; 18.1; 22.3; 24.7; 31.1; 35.0; 43.4; 43.5; 53.3; 58.4; 67.1; 67.6; 68.6; 92.3; 93.0; 93.2; 131.1; 131.4; 134.5; 140.2; 149.7; 155.3; 163.8; 164.1; 166.6; 189.0.

Lactam 190 Silyl Compound

¹H n.m.r. (300 MHz, CDCl₃) δ: 0.67; 0.93; 1.24; 1.42; 1.63; 1.84; 3.17; 3.68; 3.82; 4.10; 5.60; 7.21; 7.39. ¹³C n.m.r. (75.5 MHz, CDCl₃) δ: 7.5; 13.6; 14.5; 18.1; 18.2; 18.3; 18.6; 23.1; 23.2; 35.4; 43.4; 53.3; 58.3; 58.4; 58.5; 67.3; 69.6; 78.2; 128.9; 131.6; 132.6; 134.6; 137.6; 139.7; 155.5; 156.6; 169.3; 170.8.

Furanone 83Silyl Compound

¹H n.m.r. (300 MHz, CDCl₃) δ: 0.65; 0.88; 1.22; 1.64; 1.73; 3.16; 3.30; 4.10; 4.83; 7.39.

¹³C n.m.r. (75.5 MHz, CDCl₃) δ: 6.1; 12.3; 13.0; 16.6; 16.8; 20.8; 23.0; 29.7; 29.8; 31.2; 34.0; 41.7; 41.9; 56.8; 58.9; 67.5; 79.4; 133.3; 135.2; 147.8; 153.7; 155.1; 164.5.

Lactam 144 Silyl Compound

¹H n.m.r. (300 MHz, CDCl₃) δ: 0.63; 0.94; 1.23; 1.39; 1.62; 1.87; 3.19; 3.70; 3.81; 5.23; 5.62; 7.05; 7.24. ¹³C n.m.r. (75.5 MHz, CDCl₃) δ: 7.6; 13.7; 23.1; 35.4; 44.1; 58.4; 67.3; 69.5; 98.1; 125.9; 128.5; 131.8; 132.8; 137.5; 137.6; 139.8; 155.5; 169.7; 173.2.

### Preparation of PEG(4000)-triethyltriethoxysilyl benzenecarbamate Derivative of Furanone 83

PEG (4000) (8.0 g, 2 mmol) and (3-isocyanatopropyl)-triethoxysilane (1 g, 4 mmol) in toluene (10 mL) were heated at 85° C. with stirring for 5 h. followed by the addition of bis-(1-isocyanato-1-methylethyl)benzene (0.49 g, 2 mmol) and the mixture was stirred at 85° C. for 2 h. Furanone 83 (1.42 g, 4 mmol) was then added and the mixture further heated at 85° C. for 24 h. The mixture was cooled to r.t. and poured into light petroleum (100 mL) and stirred for 1 h. The precipitate was filtered and washed with light petroleum and dried to give a white powder (8.52 g). ¹H n.m.r. (300 MHz, CDCl₃) δ: 0.57; 0.83; 1.27; 1.64; 2.56; 3.11; 3.36; 3.60; 3.70; 4.13; 4.51; 4.73; 5.00; 5.22; 7.46. ¹³C n.m.r. (75.5 MHz, CDCl₃) δ: 7.5; 13.8; 18.1; 18.3; 22.3; 23.2; 24.6; 31.4; 32.9; 35.5; 58.0; 58.3; 61.6; 66.9; 69.5; 70.2; 71.0; 71.5; 72.4; 124.3; 134.0; 149.5; 170.0.

### Preparation of PEG(4000)-triethyltriethoxysilyl benzenecarbamate Derivative of Lactam 190

PEG (4000) (4.0 g, 1 mmol) and (3-isocyanatopropyl)-triethoxysilane (0.25 g, 1 mmol) in toluene (7 mL) were heated at 85° C. with stirring for 5 h. followed by the addition of bis-(1-isocyanato-1-methylethyl)benzene (0.30 g, 1 mmol) and the mixture was stirred at 85° C. for 2 h. Lactam 190 (0.40 g, 1 mmol) was then added and the mixture further heated at 85° C. for 24 h. The mixture was cooled to r.t. and poured into light petroleum (100 mL) and stirred for 1 h. The precipitate was filtered and washed with light petroleum and dried to give a white powder (4.21 g, 85%). ¹H n.m.r. (300 MHz, CDCl₃) δ: 0.58; 0.90; 1.19; 1.63; 1.68; 2.29; 3.13; 3.37; 3.60; 3.66; 3.78; 4.10; 4.19; 4.98; 5.22; 7.28; 7.43. ¹³C n.m.r. (75.5 MHz, CDCl₃) δ: 7.5; 12.9; 13.6; 18.2; 18.3; 22.3; 23.2; 25.0; 28.4; 29.2; 29.5; 33.0; 38.9; 43.3; 55.2; 55.3; 58.2; 60.8; 61.6; 62.0; 63.3; 63.6; 69.6; 70.2; 71.0; 72.4; 79.2; 81.4; 84.2; 111.2; 112.5; 120.8; 121.2; 122.5; 123.1; 123.7; 128.2; 128.3; 132.3; 135.4; 146.0; 146.9; 156.3; 173.9.

The following PEG 4000 silyl compounds were similarly prepared.

Furanone C6 PEG4000 Silyl Compound

¹H n.m.r. (300 MHz, CDCl₃) δ: 0.62; 0.88; 1.20; 1.65; 1.70; 2.19; 3.15; 3.63; 4.12; 4.42; 6.36. ¹³C n.m.r. (75.5 MHz, CDCl₃) δ: 7.5; 18.1; 18.2; 23.2; 28.4; 33.0; 43.3; 55.1; 57.9; 58.2; 60.7; 61.5; 64.0; 70.2; 70.4; 72.4; 78.5; 120.7; 123.6; 125.8; 128.3; 128.5; 156.3.

Furanone 116 PEG4000 Silyl Compound

¹H n.m.r. (300 MHz, CDCl₃) δ: 0.60; 0.86; 1.23; 1.65; 1.70; 2.15; 3.10; 3.60; 4.17; 7.30; 7.48. ¹³C n.m.r. (75.5 MHz, CDCl₃) δ: 7.5; 16.6; 23.2; 25.0; 29.2; 29.5; 35.5; 38.9; 43.3; 58.3; 60.0; 61.6; 62.5; 63.4; 63.6; 64.1; 66.2; 69.5; 70.2; 70.7; 72.4; 74.6; 81.0; 120.7; 122.5; 123.7; 128.3; 134.0; 198.1

Furanone 135 PEG4000 Silyl Compound

¹H n.m.r. (300 MHz, CDCl₃) δ: 0.59; 0.86; 1.20; 1.67; 2.14; 2.45; 3.61; 3.69; 3.74; 4.11; 4.52; 7.47. ¹³C n.m.r. (75.5 MHz, CDCl₃) δ: 13.9; 18.3; 22.5; 25.0; 28.9; 29.1; 31.6; 35.5; 55.3; 58.2; 67.1; 70.5; 72.4; 80.6; 123.1; 134.0; 139.9.

Lactam 144 PEG4000 Silyl Compound

¹H n.m.r. (300 MHz, CDCl₃) δ: 0.59; 0.90; 1.19; 1.63; 1.68; 2.40; 3.13; 3.36; 3.56; 3.67; 3.78; 4.11; 4.17; 5.00; 5.20; 7.03; 7.24; 7.44. ¹³C n.m.r. (75.5 MHz, CDCl₃) δ: 7.5; 18.2; 23.2; 29.3; 33.0; 28.3; 61.6; 70.5; 72.4; 120.8; 123.7; 125.9; 128.3; 128.5; 131.1; 178.2.

### Preparation of PEG (400)-triethylsilyl-triethoxybenzenecarbamate Derivative of Furanone 83

PEG (400) (1.0 g, 2.5 mmol) and (3-isocyanantopropyl)-triethoxysilane (0.62 g, 2.5 mmol) in toluene (5 mL) were heated at 85° C. with stirring for 5 h. followed by the addition of bis-(1-isocyanato-1-methylethyl)benzene (0.61 g, 2.5 mmol) and the mixture was stirred at 85° C. for 2 h. Furanone 83 (0.88 g, 2.50 mmol) was then added to the mixture and further heated at 85° C. for 24 h. The mixture was cooled to r.t. washed with n-hexane (2×100 mL). The residue was dissolved in CH₂Cl₂ (20 mL) and the solvent removed under reduced pressure to give the product as a colourless oil (1.73 g, 56%).

¹H n.m.r. (300 MHz, CDCl₃) δ: 0.89; 1.22; 1.66; 1.71; 2.34; 3.15; 3.65; 4.20; 4.54; 7.30; 7.48. ¹³C n.m.r. (75.5 MHz, CDCl₃) δ: 13.9; 18.3; 21.7; 22.4; 24.7; 31.3; 35.5; 53.3; 58.3; 61.6; 67.2; 70.2; 70.5; 72.5; 80.8; 101.4; 123.2; 134.0; 149.5; 167.1; 173.0.

### Preparation of PEG (400)-triethylsilyl-triethoxybenzenecarbamate Derivative of Lactam 190

PEG (400) (1.0 g, 2.5 mmol) and (3-isocyanatopropyl)-triethoxysilane (0.62 g, 2.5 mmol) in toluene (5 mL) were heated at 85° C. with stirring for 5 h. followed by the addition of bis-(1-isocyanato-1-methylethyl)benzene (0.61 g, 2.5 mmol) and the mixture was stirred at 85° C. for 2 h. Lactam 190 (1.0 g, 2.50 mmol) was then added to the mixture and further heated at 85° C. for 24 h. The mixture was cooled to r.t. washed with n-hexane (2×100 mL). The residue was dissolved in CH₂Cl₂ (20 mL) and the solvent removed under reduced pressure to give the product as a colourless oil (2.16 g, 67%).

¹H n.m.r. (300 MHz, CDCl₃) δ: 0.64; 0.97; 1.22; 1.66; 3.16; 3.60; 3.80; 4.17; 7.29; 7.31; 7.40. ¹³C n.m.r. (75.5 MHz, CDCl₃) δ: 10.1; 11.5; 13.7; 18.2; 18.3; 18.5; 23.2; 37.8; 53.3; 55.2; 55.3; 58.3; 58.4; 61.6; 63.4; 67.3; 69.6; 70.3; 70.5; 72.4; 101.4; 120.8; 123.7; 125.8; 128.6; 128.9; 129.3; 131.6; 139.7; 172.9.

### Preparation of PEG (400)-triethylsilyl-triethoxybenzenecarbamate Derivative of Furanone C6

PEG (400) (1.16 g, 2.91 mmol) and (3-isocyanantopropyl)-triethoxysilane (0.72 g, 2.91 mmol) in toluene (7 mL) were heated at 85° C. with stirring for 5 h. followed by the addition of bis-(1-isocyanato-1-methylethyl)benzene (0.71 g, 2.91 mmol) and the mixture was stirred at 85° C. for 2 h. Furanone 83 (1.03 g, 2.91 mmol) was then added to the mixture and further heated at 85° C. for 24 h. The mixture was cooled to r.t. and poured into light petroleum (50 mL) and the undissolved residual oil was washed with fresh light petroleum (50 mL) to give a colourless oil (2.90 g, 80%).

¹H n.m.r. (300 MHz, CDCl₃) δ: 0.56; 0.88; 1.17; 1.20; 1.61; 1.68; 3.60; 3.77; 4.17; 6.33; 7.23; 7.43. ¹³C n.m.r. (75.5 MHz, CDCl₃) δ: 7.5; 13.8; 18.1; 18.3; 22.3; 23.1; 23.2; 24.6; 24.8; 24.9; 29.3; 31.3; 31.4; 33.0; 35.0; 35.5; 43.3; 53.3; 55.2; 58.1; 58.3; 61.6; 72.2; 72.4; 93.0; 120.8; 121.2; 122.5; 123.1; 125.2; 128.1; 128.3; 128.9; 129.4; 133.5; 145.7; 146.9; 147.2; 149.7; 149.8; 154.5; 156.3; 164.1; 165.0.

The following PEG 400 silyl compounds were similarly prepared.

Furanone 116 PEG400 Silyl Compound

¹H n.m.r. (300 MHz, CDCl₃) δ: 0.61; 0.88; 1.66; 1.71; 3.15; 3.64; 3.79; 4.19; 4.54; 4.86; 5.00; 7.47. ¹³C n.m.r. (75.5 MHz, CDCl₃) δ: 7.5; 14.0; 18.3; 22.5; 23.2; 25.0; 28.4; 29.2; 29.4; 31.7; 35.5; 38.9; 43.3; 49.4; 49.5; 58.1; 58.3; 60.0; 61.6; 36.3; 63.6; 72.4; 91.5; 96.2; 101.3; 112.5; 120.7; 122.5; 123.6; 128.3; 134.0; 159.5; 173.1.

Lactam 144 PEG400 Silyl Compound

¹H n.m.r. (300 MHz, CDCl₃) δ: 0.59; 0.98; 1.22; 1.66; 1.71; 2.23; 3.17; 3.64; 3.80; 4.20; 5.26; 7.06; 7.46. ¹³C n.m.r. (75.5 MHz, CDCl₃) δ: 6.3; 13.7; 18.3; 18.5; 29.2; 29.4; 33.0; 37.9; 44.1; 53.3; 55.2; 55.3; 57.0; 58.3; 59.9; 61.7; 63.3; 67.3; 69.5; 70.3; 70.5; 72.5; 121.2; 123.1; 125.9; 127.1; 128.2; 128.5; 131.8; 139.5; 171.1.

Surface Attachment

General Procedure for Glass Slides

Glass slides (microscope cover slips) were pretreated overnight in a detergent solution followed by the following cleaning procedure.

i) rinse thoroughly with water, ii) ultrasonicate at 30° C. for 5 minutes with water, ethanol and dichloromethane, iii) ultrasonicate at 60° C. for 25 minutes with 1:1:1.5 mixture of hydrogen peroxide/ammonia/water, iv) sonicate for 25 minutes with 6:1 mixture of water/hydrochloric acid, v) rinse thoroughly with water, vi) ultrasonicate for 5 minutes each with methanol, methanol/toluene (1:1), and dry at 118° C. in an oven.

Solutions of furanone/lactams were prepared based on % w/v.

To a glass slide, mounted on a spin coater, rotating at 1000 rpm was placed 10 drops of 1% furanone/lactam (0.1 g in 10 mL toluene) followed by rotation at 2000 rpm to remove excess solution. The procedure was repeated 3 times. The slides were cured in a 95° C. over overnight followed by rinsing in toluene to remove unreacted furanone/lactam, then dried in a drying cabinet to remove the residual solvent.

XPS analysis indicated that the glass slides had been surface functionalized with the furanone/lactams.

Selected XPS Data for Coated Glass Slides

In the tables that follow:

GS means glass slide; CAT means catheter; H means hexanol (the control); S means short alkyl linker; P400 means PEG 400 linker; P4000 means PEG 4000 linker; 83, 190 etc correspond to particular compounds.

| | Sample | | | | |
|---|---|---|---|---|---|
| | GSHS | GSHP400 | GSHP4000 | GS83P4000 | GS116P400 |
| bromine | | | | 0.123 | 0.116 |
| | Sample | | | | |
| | GS190P400 | GS190S | GS83PEG400 | GS190P4000 | GSC6P400 |
| bromine | 0.175 | 4.651 | 0.395 | 0.04 | 0.532 |

General Procedure for Coating Contact Lenses

Commercial contact lenses were rinsed in Milli Q water to remove the buffered solution they were stored in, and then placed onto a paper towel prior to coating.

Solutions of furanone/lactams were prepared based on % w/v using Milli Q water as the solvent. In the case of the short linker derivatives, a 1:3 ethanol:Milli Q water mix was required to dissolve the compounds

The contact lenses were individually immersed in 1 mL of 1% furanone/lactam (0.1 g in 10 mL Milli Q water) and left on an agitator for 48 h. The solution was decanted and the contact lenses were cured at 50° C. for 48 h. Milli Q water (5 mL) was added to each lens and left for 5 h. to rehydrate the contact lenses. The contact lenses were rinsed with excess Milli Q water to remove any unreacted furanone/lactam and then stored in buffered saline solution.

XPS analysis indicated that the contact lenses had been surface functionalized with the furanone/lactams.

Attachment of Furanones to Sputter Coated Catheters

Commercial catheters (silicone rubber) were cut into 5 cm lengths and spliced down the center then pretreated by soaking in a detergent solution overnight, followed by rinsing in Milli Q water and drying in a drying cabinet. In order to functionalise the surface of the catheters with hydroxyl groups, the catheters were treated with a Bal-Tec SCD050 sputter coater at 10⁻¹ mbar under argon plasma, P=9.36 W for 120 sec. The plasma coated catheters were immersed in 1% w/v solution of furanone/lactam in Milli Q water and left on an agitator for 48 h. The solution was decanted and the catheters cured at 95° C. overnight, rinsed with Milli Q water and dried in a drying cabinet.

XPS analysis indicated that the catheters had been surface functionalized with the furanone/lactams.

Selected XPS Data for Catheters

| Sample | CAT83S | CAT83P400 | CAT83P4000 |
|---|---|---|---|
| bromine | 1.161 | 1.313 | 0.468 |

| Sample | CAT116S | CAT116P400 | CAT116P4000 |
|---|---|---|---|
| bromine | 3.785 | 0.299 | 0.096 |
| Sample | CAT190S | CAT190P400 | CAT190P4000 |
| bromine | 1.136 | 0.206 | 0.19 |

The glass coverslips were tested for their ability to reduce or prevent biofilm formation when challenged with bacteria. Three testing systems were used. The first was a short term, 24 h, batch biofilm system and the second was a once through, flow cell system, as described by {Hentzer et al., 2002, Microbiol. 148(1), 87-102}. For the batch biofilm system, the modified glass coverslips (no. 1 18×18 mm), with the attached furanones, were mounted onto glass slides as a solid support. Each slide had one furanone containing and one control coverslip. Up to six slides were placed into large, sterile glass petri dishes. An overnight culture of Gfp expressing Pseudomonas aeruginosa PAO1, or a clinical isolate of Staphylococcus aureus was inoculated into fresh media in the petri dish. Cultures were incubated for 24 h at 25° C. with shaking. The slides were rinsed three times in sterile PBS to remove loosely attached cells and were visualised by fluorescence microscopy (S. aureus cells were first stained with the BacLight Live-Dead staining kit, Molecular Probes). Biofilm formation was quantified by image analysis to determine surface coverage. Biofilm formation was normalised to the control surface, which was set at 100%. Catheter pieces, approximately 60 mm lengths, were surface modified and tested for biofilm formation by P. aeruginosa or S. aureus by pumping sterile medium through the catheter pieces for 7 days. Biofilm formation was quantified by removing the biofilm from the catheters and determining the total protein concentration.

For the once through flow cell systems, three channel flow cells were used and a treated coverglass was glued on top (no. 1, 24×60 mm). Each channel therefore, represented a replicate biofilm. Overnight cultures of Gfp expressing P. aeruginosa PAO1 were injected into the flow cells and allowed to attach. Biofilms were monitored by confocal laser scanning microscopy and images were quantified by image analysis to determine biofilm depth and surface coverage over 7 to 9 days. Results were presented as the percentage of the control coverslips, lacking furanones.

Using these testing systems, the attachment strategies described here demonstrated good biofilm inhibition. For example, treatment 190PEG400 showed approximately 50% reduction of biofilm after 24 h against P. aeruginosa. Treatment 116PEG4000 showed little or no activity against P. aeruginosa.

When biofilms of S. aureus were formed on either 83PEG400 or 83PEG4000, the total biofilm was reduced to 6% and 13% of the control respectively.

This activity is not due to killing of the cells on the surface and subsequent inhibition of colony formation through clonal growth. Comparison of the percentage of live and dead cells on the surface indicated that there was no difference in the ratio of live and dead cells on the furanone treated surfaces (FIG. 1).

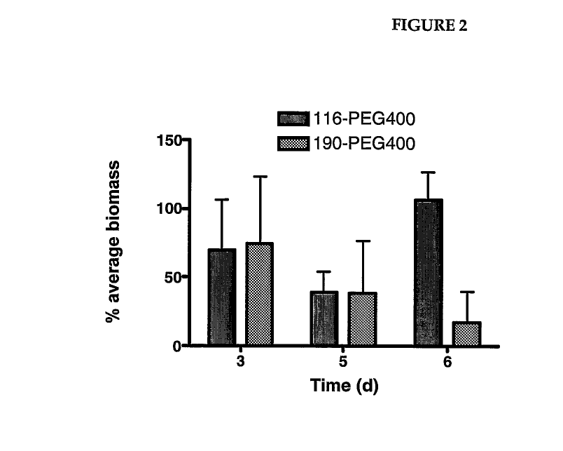

Using the flow cell systems, biofilm formation of P. aeruginosa on furanone containing surfaces was evaluated. These data show that the furanone modified surfaces inhibit biofilm formation by P. aeruginosa over the period tested. For example, treatments 83PEG4000 and 116S1 had an average biofilm reduction of 50% over 7 days. Treatment 190PEG400 reduced biofilm by 50% up to day 5 and 75% on day 6, but showed no difference at day 7 (data not shown) suggesting the surface treatment was overwhelmed after 4 days (FIG. 2). Treatment 116PEG400 showed a maximum of 50% inhibition, but this inhibition was lost by day 6 (FIG. 2). Treatment 190PEG4000 showed up to 75% inhibition of biofilm formation.

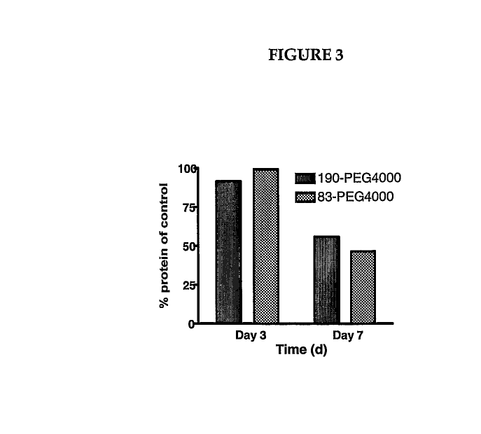

Biofilm formation by P. aeruginosa and S. aureus on modified catheters was also monitored. Treatments 190S1 and 116S1 significantly reduced P. aeruginosa biofilm formation, while 83PEG400 showed reduced biofilm formation on day 3 but not on day 7 (data not shown). Compounds 190PEG4000 and 83PEG4000 showed approximately 50% reduction in S. aureus biofilm formation on day 7, but no difference on day 3 (FIG. 3).

In order that the nature of the present invention may be more clearly understood, preferred forms thereof will now be described with reference to the following non-limiting examples.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed anywhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An antimicrobial silicone oligomer or polymer comprising a silicone oligomer or polymer associated with at least one compound of formula I 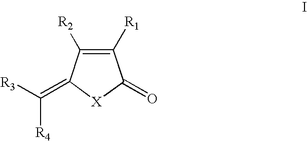
wherein, R1 and R2 are independently selected from the group consisting of H, halogen, alkyl, alkoxy, oxoalkyl, alkenyl, aryl or arylalkyl whether unsubstituted or substituted, straight chain or branched chain,
R3 and R4 are independently H, halogen, alkyl, aryl, or arylalkyl; and X is O or NR₂.

2. A antimicrobial silicone oligomer or polymer according to claim 1 wherein R1 and R2 of the compound of formula I are independently hydrophobic, hydrophilic or fluorophilic.

3. An antimicrobial silicone oligomer or polymer according to claim 1 wherein at least one of R1, R2, R3 and R4 is a halogen.

4. An antimicrobial silicone oligomer or polymer according to claim 3 wherein at least one of R1, R2, R3 and R4 is bromine.

5. An antimicrobial silicone oligomer or polymer according to claim 1 wherein the compound of formula I is blended or mixed with the silicone oligomer or polymer.

6. An antimicrobial silicone oligomer or polymer according to claim 1 wherein the compound of formula I is adsorbed to the silicone polymer or oligomer.

7. An antimicrobial silicone oligomer or polymer according to claim 6 wherein the compound of formula I is adsorbed to the silicone polymer or oligomer by direct application of the compound of formula I to the silicone polymer or oligomer.

8. An antimicrobial silicone oligomer or polymer according to claim 1 formed by copolymerising a compound of formula I with at least one silicone comonomer or oligomer and optionally at least one other monomer.

9. An antimicrobial silicone oligomer or polymer according to claim 1 formed by condensation polymerisation of a silicone monomer or oligomer or polymer with the compound of formula I.

10. An antimicrobial silicone polymer or oligomer according to claim 1 formed by surface attachment of a compound of formula I on to a silicone polymer or oligomer or a device formed at least in part therefrom.

11. An antimicrobial polymer or oligomer according to claim 10 wherein the silicon polymer or oligomer or the device is chemically or plasma treated.

12. An antimicrobial silicone oligomer or polymer according to claim 1 wherein the compound of formula I is a compound of formula II 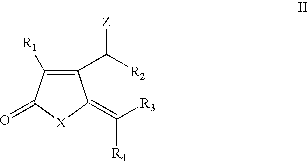
wherein R₁, R₂ are independently selected from H, alkyl, alkoxy, polyethyleneglycol, oxoalkyl, alkenyl, aryl or arylalkyl whether unsubstituted or substituted, straight chain or branched chain;
R₄ is a hydrogen, halogen (X═F, Cl, Br or I);
R₃ is hydrogen or halogen; and
X is O or NR₂ and
Z is independently selected from the group R₂, halogen, OH, OOH, OC(O)R₂, ═O, amine, azide, thiol, mercaptoalkyl, mercaptoalkenyl, alkenyloxy, aryloxy, mercaptoaryl, arylalkoxy, mercaptoarylalkyl, SC(O)R₂, OS(O)₂R₂, NHC(O)R₂, ═NR₂, NHR₂ or silyloxy.

13. A antimicrobial silicone oligomer or polymer according to claim 12 wherein R1 and R2 of the compound of formula I are independently hydrophobic, hydrophilic or fluorophilic.

14. An antimicrobial silicone oligomer or polymer according to claim 1 wherein the silicone oligomer or polymer is selected from the group comprising hexamethyldisioxane, octamethyltrisioxane, decamethyltetrasioxane, dodecamethylpentasiloxane, tetradecamethylhexasiloxane, hexamethyltricyclosiloxane, decamethylpentacyclosiloxane, dodecamethylhexacyclosiloxane, and dimethylpolysiloxane.

15. A compound of formula III: 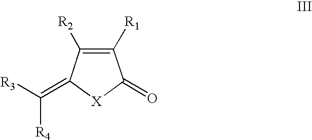
wherein, R1 and R2 are independently selected from the group consisting of H, halogen, alkyl, alkoxy, oxoalkyl, alkenyl, aryl or arylalkyl whether unsubstituted or substituted, straight chain or branched chain,
R3 and R4 are independently H, halogen, alkyl, aryl, or arylalkyl; and X is O or NR₂,
wherein the compound of formula III has at least one —YC(O)NR₇R₅Si(OR₆)₃ group, where Y is selected from the group O, S, N, P, C(O); R₅ is a linker and preferably is substituted or unsubstituted alkyl, alkylaryl, arylalkyl, aryl, alkenyl, or a linker comprising these groups, optionally interrupted by one of more heteroatoms (eg oxygen), or a linking group comprising these groups and each R₆ is independently selected from substituted or unsubstituted alkyl, cycloalkyl, alkenyl or the like and R₇ is H or alkyl.

16. A compound according to claim 15 wherein the linker R5 is a polyoxoalkylene.

17. A antimicrobial silicone oligomer or polymer according to claim 1 wherein R1 and R2 of the compound of formula I are independently hydrophobic, hydrophilic or fluorophulic.

18. A method of producing a compound according to formula III of claim 15, comprising reacting a compound of formula I having at least one group selected from —Y′—H, wherein —Y′ is selected from the group O, S, NH, COO with a compound of formula OCNR₇R₅Si(OR₆)₃, wherein R₅ is a linker and preferably is substituted or unsubstituted alkyl, alkylaryl, arylalkyl, aryl, alkenyl, or a linker comprising these groups, optionally interrupted by one of more heteroatoms (eg oxygen), or a linking group comprising these groups and each R₆ is independently selected from substituted or unsubstituted alkyl, cycloalkyl, alkenyl or the like and R₇ is H or alkyl.

19. A method for associating a compound of formula III of claim 15 with a surface, the method comprising contacting the compound of formula III with the surface and optionally curing the compound.

20. A method according to claim 19 wherein prior to the step of contacting the compound of formula III with the surface, the surface is treated to produce groups that are reactive with the silyloxy group of the compound of formula III.

21. A method for associating a compound of formula III according to claim 15 with a polymer or oligomer; the method comprising contacting the compound of formula III with the surface and optionally curing the polymer or oligomer.

22. A method according to claim 21 wherein the polymer or oligomer is a silicone polymer or oligomer.

23. A polymer or oligomer associated with a compound of formula III according to claim 15.

24. A polymer or oligomer of claim 23 wherein the polymer or oligomer is a silicone polymer or oligomer.

25. A compound of formula III according to claim 15 wherein the compound is of formula IV: 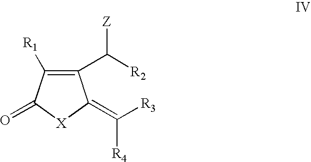
wherein R₁, R₂ are independently selected from H, alkyl, alkoxy, polyethyleneglycol, oxoalkyl, alkenyl, aryl or arylalkyl whether unsubstituted or substituted, straight chain or branched chain;
R₄ is a hydrogen, halogen (X═F, Cl, Br or I);
R₃ is hydrogen or halogen; and
X is O or NR₂ and
Z is independently selected from the group R₂, halogen, OH, OOH, OC(O)R₂, ═O, amine, azide, thiol, mercaptoalkyl, mercaptoalkenyl, alkenyloxy, aryloxy, mercaptoaryl, arylalkoxy, mercaptoarylalkyl, SC(O)R₂, OS(O)₂R₂, NHC(O)R₂, ═NR₂ NHR₂ or silyloxy;
wherein the compound of formula IV has at least one —YC(O)NR₇R₅Si(OR₆)₃ group, where Y is selected from the group O, S, N, P, C(O); R₅ is a linker and preferably is substituted or unsubstituted alkyl, alkylaryl, arylalkyl, aryl, alkenyl, or a linker comprising these groups, optionally interrupted by one of more heteroatoms (eg oxygen), or a linking group comprising these groups and each R₆ is independently selected from substituted or unsubstituted alkyl, cycloalkyl, alkenyl or the like and R₇ is H or alkyl.

26. A device coated with a compound of formula III according to claim 15 or with a compound of formula IV as defined above.

27. A device according to claim 26 wherein the device is a contact lens.

28. A device according to claim 26 wherein the device is a catheter.

29. A device according to claim 26 wherein the device is a separation membrane used for water treatment.

30. A device according to claim 26 wherein the device is a bandage.

31. A device according to claim 26 wherein the device is an alginate bead.
